(19) United States
(12) Patent Application Publication
(10) Pub. No.: US 2006/0276423 A1
(43) Pub. Date: Dec. 7, 2006

(54) Survivin-directed RNA interference-compositions and methods

(76) Inventors: Rachel Altura, Bexley, OH (US); Hugo Caldas, Columbus, OH (US); Michael Holloway, Bexley, OH (US)

Correspondence Address: HOWREY LLP, C/O IP DOCKETING DEPARTMENT, 2941 FAIRVIEW PARK DR, SUITE 200, FALLS CHURCH, VA, 22042-2924, US

(21) Appl. No.: 11/406,740

(22) Filed: Apr. 18, 2006

Related U.S. Application Data
(60) Provisional application No. 60/672,417, filed on Apr. 18, 2005.

(51) Int. Cl. A61K 48/00; A61K 31/337; C12Q 1/68; C07H 21/02

(52) U.S. Cl. 514/44; 514/449; 435/6; 536/23.2

(57) ABSTRACT

The present invention is directed to compositions and methods for inhibiting the expression of survivin in cells expressing survivin. The invention is also directed to methods of treating conditions associated with elevated survivin, such as hyperproliferative disorders. More particularly, the invention is directed to inhibition of survivin expression using short interfering RNAs (si-RNAs) or through administration of DNA sequences yielding the expression of short hairpin RNAs (sh-RNAs).

### CROSS REFERENCE TO RELATED APPLICATIONS

The present application claims the benefit of U.S. Provisional Application No. 60/672,417, filed Apr. 18, 2005, which is incorporated herein by reference in its entirety.

### FIELD OF THE INVENTION

The present invention is directed to the field of oncology and other disease-states, such as rheumatoid arthritis, pulmonary hypertension and atherosclerosis and more specifically it provides compositions and methods for treating these diseases. More particularly, the present invention is directed to compositions and methods for promoting apoptosis in diseased cells. The invention is also directed to compositions and methods for inhibiting the anti-apoptotic activity of Survivin, and therefore, may be used for stimulating apoptosis in human tissues and cell populations that express Survivin.

### BACKGROUND OF THE INVENTION

1. Expression of Survivin by Patient's Cancer Cells Correlates with Poor Survival Prognosis for the Patient.

Apoptosis is an active physiological process that results in self-destruction of a targeted cell (Brown et al., 2005). The apoptotic process can be initiated by a variety of regulatory stimuli and serves as a safeguard mechanism by which a multicellular organism eliminates improperly proliferating cells (Brown et al., 2005). Changes in the cell survival rate play an important role in human pathogenesis of diseases, e.g. cancer cells often become unresponsive to regulatory stimuli that promote apoptosis in normal cells (Brown et al., 2005).

A variety of chemotherapeutic compounds and radiation induce apoptosis in tumor cells, in many instances via wild-type p53 protein. However, about 50% of cancers do not respond well to treatments with currently available chemotherapeutic compounds or radiation. The failure to respond usually results from inability of the chemotherapeutic compounds to trigger apoptosis in the cancer cells. Thus, new methods and compositions are needed to trigger apoptosis in cancer cells.

Survivin is a gene with structural and functional similarities to both the inhibitor-of-apoptosis gene family, which specifically blocks the downstream effectors of cell death, and to the chromosomal passenger proteins, which play an essential role in cytokinesis (Li et al., 1998; Li et al., 1999; Uren et al., 2000; Skoufias et al., 2000). The human Survivin gene locus encodes at least five alternatively spliced transcripts including Survivin, Survivin-2B, Survivin-ΔEx3, Survivin-3B, and Survivin-2alpha (Conway et al., 2000; Mahotka et al., 1999; Konno et al., 2000; Caldas et al., 2005). Survivin is highly expressed during normal embryonic development and in a variety of transformed cell lines, but minimally expressed in normal, non-transformed tissue. Adult tissues that do express survivin include those composed of highly proliferating cells such as bone marrow, vascular endothelium, endometrium and neural stem cells (Konno et al., 2000; Fukuda et al., 2001; Altura et al., 2003).

Survivin's role in cell proliferation is primarily as a chromosomal passenger protein, ensuring proper nuclear and cytoplasmic division both directly and indirectly by interacting with partner proteins. Treatment with survivin inhibitors result in an aberrant localization of Aurora B kinase during assembly of the anaphase-promoting complex (Chen et al., 2003; Honda et al., 2003). As a consequence, the cell loses its ability to phosphorylate critical mitotic targets, such as histone H3B, vimentin fibers at the cleavage furrow, kinesin-like motor protein, spindle apparatus proteins, kinetochore protein and the tumor suppressor protein p53 (Wheatley et al., 2004; Ota et al., 2002; Katayama et al., 2003).

Survivin inhibits apoptosis by interacting directly or indirectly with downstream proteins, thereby inhibiting activation of apoptotic signaling cascades. In addition to its inhibition of Smac/DIABLO (Song et al., 2003), a pro-apoptotic mitochondrial protein, mitochondrial Survivin inhibits the processing of caspase-3 and caspase-9 proteins to their active forms (Dohi et al., 2004).

Human cancer cells that aberrantly express Survivin include epithelial tumors of lung, colon, pancreas, breast, stomach, CNS tumors, soft tissue sarcomas and hematologic malignancies (reviewed in Altieri, 2003). Pediatric tumors that express Survivin include neuroblastoma, Wilms' tumors, and some pediatric CNS malignancies (Altura et al., 2003; Fangusaro et al., 2005). U.S. Pat. No. 6,800,737 discloses that most of the cancers express Survivin. High levels of expression within cancer cells have correlated with clinical outcome in many studies; the majority of these studies showing a direct correlation between higher levels of Survivin and a poor outcome (Altieri, 2003). In agreement with this observation, U.S. Pat. No. 6,656,684 B1 and U.S. patent application Ser. No. 10/291,607 disclose that high levels of survivin detected in patient's biological fluid are indicative of cancer recurrence.

Disruption of survivin within several types of cancer cells enhances programmed cell death, while overexpression of this gene inhibits apoptosis (Altieri, 2003). There are several US patents and patent applications pertinent to methods of blocking Survivin expression in a cell. U.S. Pat. No. 6,800,737 and U.S. patent application Ser. No. 10/354,090 disclose antibodies specific to human Survivin that block Survivin's anti-apoptotic functions, while U.S. patent application Ser. No. 10/807,897 discloses DNA vaccines that may be used for stimulating an immune response in a patient against Survivin. In addition, U.S. Pat. Nos. 6,608,108 and 6,777,444 disclose chemical compounds that block Sp1-dependent gene expression and may be used for blocking Survivin's expression by interfering with its promoter activity. U.S. Pat. Nos. 6,335,194 and 6,838,283 teach that Survivin's expression in cells and tissues can be inhibited by anti-sense compound 8 to 30 nucleotides in length complementary to Survivin.

2. Short Interfering RNA (si-RNA)

Recently a cellular process that eliminates selected RNA transcripts has been identified (Brummelkamp et al., 2002) On the basis of this process, a technology, called short interfering RNA technology (si-RNA technology), has been developed. In the si-RNA technology, short interfering RNA (si-RNA) with partial or fully double-stranded character is introduced into a cell or into the extracellular environment. The introduced si-RNA is designed in such a way that a portion of the si-RNA contains a sequence complementary to a gene whose expression is to be eliminated in the cell. The si-RNA then specifically targets transcripts of the gene for degradation by what is known as the RISC pathway.

U.S. patent application Ser. No. 10/685,837, entitled “si-RNA mediated gene silencing in transgenic animals”, incorporated herein by reference in its entirety, teaches that gene expression can be silenced in a living organism by using a short hairpin RNA (sh-RNA) expression vector integrated into genome of the organism. Sh-RNAs comprise a single stranded RNA loop and a double stranded RNA stem. The stem portion of sh-RNA may be about 30 nucleotides long and comprises a sequence complementary to a gene whose expression is to be silenced

### SUMMARY OF THE INVENTION

Provided are compositions and methods of inhibiting the expression of survivin in any cells or tissues that express survivin, by means of introducing into cells si-RNAs or sh-RNAs that selectively inhibit expression of Survivin mRNAs. Also provided are compositions and methods of eradicating survivin-expressing cancers, by means of introducing into cells si-RNAs or sh-RNAs that selectively inhibit expression of Survivin m-RNAs and trigger Programmed Cell Death (PCD) in the targeted cells.

In one embodiment, the instant invention is directed to compositions comprising short-interfering RNA (si-RNA) that is at least 50% identical and up to 100% identical to si-RNA with SEQ ID NOs: 1, 2, 3 or 4. For example, the si-RNA may be 50%, 55%, 60%, 65%, 70%, 75%, 80%, 85%, 90%, 95% or 100% identical to si-RNA with SEQ ID NOs: 1, 2, 3 or 4. The compositions of the instant invention may contain several si-RNAs, each of which is at least 50% identical to si-RNA with SEQ ID NOs: 1, 2, 3 or 4. Other compositions of the instant invention include compositions comprising si-RNA with SEQ ID NO: 1, 2, 3 or 4. Yet other compositions of the instant invention include mixtures of any two of the four si-RNAs, any three of the four si-RNAs or a mixture of the four si-RNAs.

In another embodiment, the instant invention teaches compositions comprising vectors that after delivery into targeted cells express sh-RNA such that the si-RNA portion of sh-RNA is at least 50% identical to si-RNA with SEQ ID NOs: 1, 2, 3 or 4. For example, the si-RNA portion may be 50%, 55%, 60%, 65%, 70%, 75%, 80%, 85%, 90%, 95% or 100% identical. The compositions of the instant invention may contain one or more vectors expressing several different sh-RNAs, each of which is at least 50% identical to si-RNA with SEQ ID NOs: 1, 2, 3 or 4. Other compositions of the instant invention include compositions comprising an expression vector containing sh-RNA in which the si-RNA portion is selected from those having SEQ ID NO: 1, 2, 3 or 4. Yet other compositions of the instant invention include mixtures of expression vectors for any two of the three si-RNAs, any three of the four si-RNAs or a mixture of expression vectors for the four si-RNAs.

The instant invention is also directed to compositions comprising DNA sequences encoding sh-RNAs of the instant invention, and methods of using the compositions. DNA sequences encoding sh-RNAs of the instant invention comprise three regions: the first region comprises a 15 to 35, preferably a 19 to 29 base pair DNA sequence at least 50%, preferably 100% homologous to Survivin having SEQ ID NOs 22, 23 or 24; the second of the regions comprises a spacer DNA sequence having 5 to 9 base pairs forming the loop of the expressed RNA hair pin molecule; and the third of the regions 15 to 35, preferably 19 to 29 base pair DNA sequence at least 85% complementary to the first region.

Each of the compositions of the instant invention can be used in combination with any other anti-cancer agents, such as any chemotherapeutical agents known in the art, peptides or compositions developed via gene therapy approaches.

The compositions of the instant invention can be delivered by any known method of administration including intraperitoneal, oral, intranasal, parenteral, intrathecal, intraventricular, and injection, including a hydrodynamic injection into a tumor.

In another embodiment, the instant invention is directed to methods of inhibiting the expression of survivin in cells or tissues that express survivin comprising contacting the cells or tissues with any of the compositions of the instant invention. The expression of survivin in said cells or tissues may be appropriate or aberrant. Representative examples of cells that appropriately express survivin include stem cells, progenitor cells, bone marrow cells, vascular endothelial cells and endometrial cells.

The instant invention is also directed to methods of treating conditions associated with elevated survivin expression comprising administering to a subject in need of such treatment any of the compositions of the instant invention. Conditions associated with elevated survivin expression that may be treated with compositions of the instant invention include cardiovascular disease and auto-immune disease.

In yet another embodiment, the instant invention is also directed to methods of treating cancers or other hyperproliferative disorders, by administering to a subject in the need of such treatment any of the compositions of the instant invention. The methods of the instant invention can be used to treat, for example, lung cancer cells, colon cancer cells, pancreatic cancer cells, breast cancer cells, stomach cancer cells, central nervous system cancer cells, soft tissue sarcoma cells, hematologic malignant cells or pediatric cancer cells under the conditions where the cancer cells express survivin.

The instant invention is also directed to methods for diagnosing cancers and tumors that may be treated with the compositions of the instant invention. Specifically, according to the teachings of the instant invention, cancers and tumors that express survivin can be treated with the compositions of the instant invention. The diagnostic methods of the instant invention are based on performing quantitative polymerase chain reaction (PCR) analysis of RNA from a patient's tumor or cancer tissue and control healthy tissue using oligonucleotides selected from a group consisting of oligonucleotides with SEQ ID NOs. 13, 14, 15, 16, 17, 18, 19, 20 and 21 as primers to measure the level of expression of survivin; comparing the levels of expression in the cancer sample with the healthy control; and concluding that if the cancer sample expresses survivin, then the cancer can be treated with si-RNAs or sh-RNAs of the instant invention. The diagnostic method may also be performed with oligonucleotides at least 60% identical to oligonucleotides with SEQ ID NOs. 13, 14, 15, 16, 17, 18, 19, 20 and 21.

The instant invention is also directed to prognostic methods that aid in determining whether a patient's cancer may be resistant to chemotherapy and irradiation. According to the teachings of the instant invention, patients whose tumors express survivin are patients with a poor survival prognosis in part because such tumors are typically resistant to standard treatment methods such as chemotherapy and irradiation. The prognostic methods of the instant invention are based on performing a quantitative polymerase chain reaction analysis of RNA from the patient's tumor or cancer tissue and control healthy tissue with oligonucleotides selected from a group consisting of oligonucleotides with SEQ ID NOs. 13 through 21; comparing the levels of expression in the cancer sample with the healthy control; and concluding that if the cancer sample expresses survivin at the levels higher than the control sample, then the cancer may be resistant to chemotherapy and irradiation.

The instant invention is also directed to methods for increasing the sensitivity of a cancer cell to chemotherapeutic agents by administering compositions of the instant invention comprising an si-RNA or sh-RNA which down regulates expression of Survivin. The si-RNA or sh-RNA may comprise sequence at least 50% identical to SEQ ID NO. 24. The chemotherapeutic agent may be taxol or any other chemotherapeutic agent known in the art. The cancer cell may be resistant to the chemotherapeutic agent. Alternatively, the cancer cell may be sensitive to the chemotherapeutic agent.

The instant invention is also directed to methods for inhibiting angiogenesis. Specifically, according to the teachings of the instant invention, angiogenesis may be inhibited with compositions of the instant invention comprising an si-RNA or sh-RNA that down regulates expression of Survivin ΔEx3. The si-RNA or sh-RNA may comprise sequence at least 50% identical to SEQ ID NO. 24. The methods of the instant invention may also be used to treat any condition associated with angiogenesis. Also provided is a vector encoding an sh-RNA wherein the si-RNA portion comprises sequence at least 50% identical to SEQ ID NO. 24. Compositions of the method may be administered by any known method of administration including those mentioned previously.

### BRIEF DESCRIPTION OF THE DRAWINGS

FIG. 1: Reduction of Survivin and survivin splice variants in response to shRNA treatment in vitro. (A) Levels of Survivin gene family transcripts were decreased after 48 hours of treatment with shRNA cocktail. (B) Levels of Survivin and Survivin ΔEx3 proteins were decreased after 48 hours of treatment with survivin shRNA cocktail.

FIG. 2: Schematic representation of Survivin gene structure and targeting with shRNAs. Grey boxes represent exons. The survivin gene contains exons 1-4; survivin-2B contains exons 1-4 and an additional exon 2B; survivin ΔEx3 contains exons 1, 2 and 4. The black lines and arrows above the figure show regions targeted by the different survivin shRNAs.

FIG. 3: Inhibition of Survivin by shRNA cocktail induces tumor cell death. RMS cells were transfected with Survivin shRNAs for a minimum of 24 hours. The cells were sorted for an enriched cell population. Growth of cells was assessed by trypan blue exclusion at 24-hour intervals. (A) shRNA-treated cells grew slower than control-treated cells, (B) had higher levels of apoptosis, as assayed by annexin V staining and (C) showed activation of caspase 3.

FIG. 4: Survivin shRNA cocktail inhibits tumor growth in vivo. NOD/SCID mice subcutaneously injected with RH30^{red} red cells formed palpable tumors within 21 days. Mice were randomly assigned treatment groups (control or treated) and injected with shRNA cocktail or pSUPER, as described in the methods. Tumor growth is represented as increase over baseline.

FIG. 5: Survivin cDNA sequences.

FIG. 6: Sensitization of taxol-resistant tumor cells to taxol treatment following transfection with Survivin-ΔEx3 shRNA. Tumor cell lines were transfected with Survivin-ΔEx3 shRNA or scrambled shRNA with or without Taxol (20 μM). Cells were incubated for 24, 48 or 72 hours post transfection and assayed for apoptosis. The graph shows that tumor cells resistant to taxol treatment become sensitive to taxol treatment following transfection with Survivin-ΔEx3 shRNA and that tumor cells sensitive to taxol exhibit an additive effect from treatment in combination with Survivin-ΔEx3 shRNA

FIG. 7: Interference with Survivin-ΔEx3 inhibits angiogenesis in vitro. Human Umbilical Vein Endothelial (HUVE) cells were transfected with shRNAs of the instant invention or incubated with antibodies against Survivin-ΔEx3 and the results compared to HUVE cells treated with known inducers/inhibitors of in vitro angiogenesis. Treated/transfected cells were plated on growth factor reduced basement membrane extract (BME) to support in vitro tube formation. (C) shows that treatment with shRNA that down regulate Survivin-ΔEx3 resulted in impaired ability to form tubes as compared with a scrambled shRNA and a survivin shRNA not targeting Survivin-ΔEx3. (D) shows that incubation of HUVE cells with anti-Survivin-ΔEx3 antibody (10 μg/ml) resulted in impaired ability to form tubes as compared to an isotype control antibody (10 μg/ml). (B) shows the effects of treating HUVE cells with soluble VEGF, anti-VEGFR1, anti-VEGFR2 antibodies or actinomycin-D, known inducers and inhibitors of angiogenesis. Quantitative data shown in (E) are mean±s.e. of 5 replicates per sample.

FIG. 8: Interference with Survivin-ΔEx3 inhibits angiogenesis in vivo. (A) Angioreactors filled with extracellular matrix pre-mixed with angiogenic factors (heparin, FGF-2) containing either no antibody (positive control), normal rabbit IgG or Survivin-ΔEx3-specific antibody were implanted subcutaneously into the dorsal flank of athymic nude mice, then removed 11 days post-implantation. Angioreactors filled with extracellular matrix without angiogenic factors served as a negative control. (B) shows that vascularization of the angioreactor was virtually absent in the Survivin-ΔEx3 antibody treated and negative control samples in contrast to the prominent vascular growth in the positive control and IgG samples. (C) depicts a graph representing the mean fluorescence±s.e. from 4 replicates

### DETAILED DESCRIPTION

The terminology used herein is for the purpose of describing particular embodiments only and is not intended to be limiting. As used in the specification and the appended claims, the singular forms “a,” “an” and “the” include plural referents unless the context clearly dictates otherwise.

A “small interfering RNA” or siRNA comprises a short double-stranded RNA portion wherein the nucleotide sequence of the double-stranded portion of the siRNA is complementary to a nucleotide sequence of a targeted gene. The siRNA may comprise sequence at least 50% identical and up to 100% identical to the nucleotide sequence of the targeted gene. For example, the siRNA may comprise sequence 50%, 55%, 60%, 65%, 70%, 75%, 80%, 85%, 90%, 95% or 100% identical or any other subrange or specific value within the interval between 60% and 100%. The siRNA optionally further comprises one or two single-stranded overhangs, e.g., 3′ and/or 5′ overhangs. The double-stranded portion is approximately 15 to 35 nucleotides in length. For example, the duplex portion may be 19-29 nucleotides in length or any other subrange or specific value within the interval between 15 and 35. The double stranded portion of the siRNA molecule can be part of a hairpin structure, as in a short hairpin RNA (shRNA). In addition to the double stranded portion, the hairpin may contain a loop portion positioned between the two sequences that form the double stranded portion. In some embodiments the loop is 5 to 20 nucleotides in length. The hairpin structure can also contain 3′ or 5′ overhang portions.

As described below, the instant invention is directed inter alia to the use of si-RNA technology to inhibit expression of Survivin, which is useful in treating cancers that express and other diseased cells that express Survivin. The instant invention is also directed inter alia to the use of expression vectors that express sh-RNAs in targeted cells and inhibit expression of Survivin in the cells.

In one preferred embodiment, the instant invention is directed to methods of identifying cancers that express survivin. According to the teachings of the instant invention, such survivin-positive cancers can be identified either by measuring levels of survivin protein expression or levels of survivin m-RNA expression.

The instant invention provides compositions and methods for analyzing cancers of any type for expression of survivin. Examples of cancers include, but are not limited to, solid tumors and hematologic malignancies, including: malignant carcinoid syndrome, carcinoid heart disease, carcinoma (e.g., Walker, basal cell, basosquamous, Brown-Pearce, ductal, Ehrlich tumor, in situ, Krebs 2, Merkel cell, mucinous, non-small cell lung, oat cell, papillary, scirrhous, bronchiolar, bronchogenic, squamous cell, and transitional cell), histiocytic disorders, leukemia (e.g., B cell, mixed cell, null cell, T cell, T-cell chronic, HTLV-II-associated, lymphocytic acute, lymphocytic chronic, mast cell, and myeloid), histiocytosis malignant, Hodgkins disease, Non-Hodgkins lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewings sarcoma, synovioma, adenofibroma, adenolymphoma, carcinosarcoma, chordoma, cranio-pharyngioma, dysgerminoma, hamartoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcoma (e.g., Ewings, Kaposi, and mast cell), neoplasms (e.g., bone, breast, digestive system, colorectal, liver, pancreatic, pituitary, testicular, orbital, head and neck, central nervous system, acoustic, pelvic, respiratory tract, and urogenital), neurofibromatosis, and cervical dysplasia.

Expression of survivin protein may be evaluated by immunohistochemical staining with a polyclonal or monoclonal antibody specific to survivin protein. Levels of survivin protein expression can also be measured by Western blot analysis or by immunoprecipitation with a survivin-specific antibody. One of such survivin-specific antibodies suitable for the methods of the instant invention is a polyclonal anti-survivin antibody commercially available from Santa Cruz company under company's catalog number FL-142. Monoclonal survivin-specific antibodies and methods of their preparation have been disclosed in U.S. Pat. No. 6,800,737.

A quantitative PCR analysis may be used to assess the expression of survivin in a particular cancer type. To perform this analysis, total RNA may be isolated using, for example, the Trizol® method. Other methods for total RNA isolation well known to a person skilled in the relevant art may also be used. cDNA may be obtained from the total RNA samples in a random priming reaction using Omniscript® reverse transcriptase (Qiagen). Other methods for preparing cDNA well known to a person of relevant skill may also be used. The cDNA may be then used for a quantitative PCR to determine comparative levels of survivin message in cancer samples.

The invention is also directed to survivin specific primers and preferably to the following survivin-specific primers which can be used in a quantitative PCR analysis:

Survivin specific primers:

(Seq ID NO: 13)

forward 5′ GTG AAT TTT TGA AAC TGG ACA GAG AAA;

(Seq ID NO: 14)

reverse 5′ CAC TTT CTT CGC AGT TTC CTC AA;

(Seq ID NO: 15)

probe 5′ FAM AGC CAA GAA CAA AAT TGC AAA GGA AAC
CA;
Survivin-2B splice variant specific primers:

(Seq ID NO: 16)

forward 5′ GCA CGG TGG CTT ACG CCT G;

(Seq ID NO: 17)

reverse 5′ ACC GGA CGA ATG CTT TTT ATG TTC C;

(Seq ID NO: 18)

probe 5′ FAM ATA CCA GCA CTT TGG GAG G;
Survivin-ΔEx3 splice variant specific primers:

(Seq ID NO: 19)

forward 5′ GCT GGG AGC CAG ATG ACG;

(Seq ID NO: 20)

reverse 5′ TTC GCA GTT TCC TCA AAT TCT TT;

(Seq ID NO: 21))

probe 5′ FAM CCC CAT GCA AAG GAA ACC AAC AAT AAG
AA.

In addition to a quantitative PCR analysis, any of the survivin-specific primers with SEQ ID NOs: 13 to 21 may also be used for preparing survivin-specific probes and in such RNA detecting methods as RT-PCR, in situ hybridization, Northern blot analysis or any other RNA analysis.

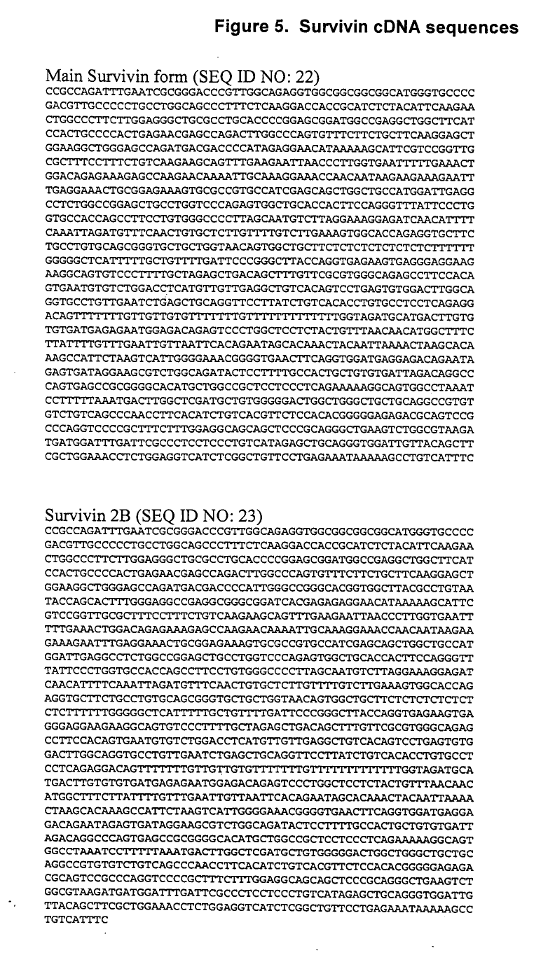

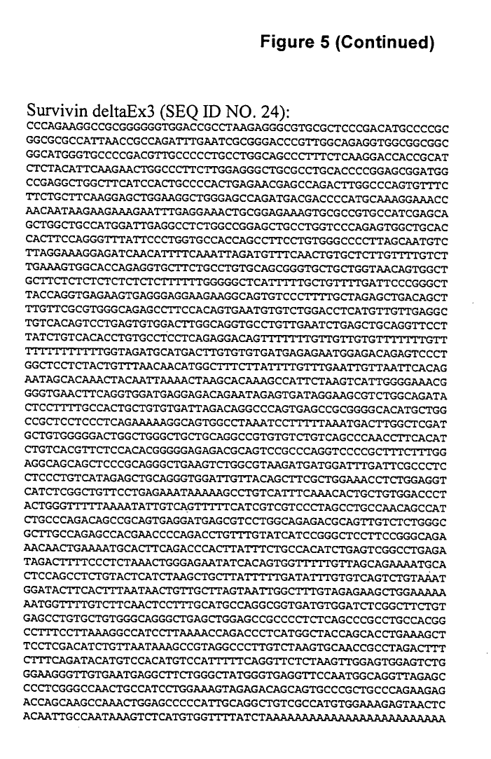

The invention is also directed to Survivin cDNAs. The following cDNA sequences are disclosed herewith in FIG. 5: cDNA sequence for main Survivin form (SEQ ID NO: 22); cDNA for Survivin 2B (SEQ ID NO: 23) and cDNA for Survivin delta Ex3 (SEQ ID NO: 24). U.S. Pat. No. 6,800,737, incorporated herein by reference, discloses amino acid and cDNA sequence of human survivin. Full length cDNA sequence for main Survivin form and for Survivin 2B is provided as SEQ ID NOs: 25 and 26 respectively.

In another embodiment, the instant invention is directed to methods of inhibiting the expression of survivin in cells or tissues that express survivin by contacting the cells or tissues with compositions of the instant invention. The expression of survivin in the cells or tissues may be appropriate or aberrant. Survivin is appropriately expressed in several highly proliferative areas within normal tissues. For example, survivin is expressed in vascular endothelial cells, hematopoietic cells, stem cells such as neural stem cells, bone marrow cells, and endometrial cells. Compositions of the instant invention may be used to inhibit the expression of survivin in these cells.

Also provided are methods of treating conditions associated with elevated expression of survivin comprising administering to a subject in need thereof, an effective amount of any of the compositions of the instant invention. One example of a condition associated with elevated expression of survivin is an auto-immune disease such as rheumatoid arthritis. Another condition associated with elevated expression of survivin is a cardiovascular disease such as pulmonary arterial hypertension or atherosclerosis. Administering an effective amount of compositions of the instant invention to a subject in need thereof, treats a condition associated with elevated expression of survivin by down regulating the expression of survivin. The amenability of a cell or tissue from a candidate subject to treatment with compositions of the instant invention may be determined by measuring the level of survivin expression in the subject's cell or tissue with comparable cell or tissue from a healthy control, comparing the level of survivin expression in the subject's cell or tissue with that of the healthy control and determining that an elevated level of survivin expression in the candidate subject's cell or tissue compared to the level of survivin expression in the healthy control indicates that a candidate subject's cell or tissue is amenable to treatment with compositions of the instant invention.

In yet another embodiment, the instant invention is directed to methods of treating survivin-expressing cancers. Any survivin-expressing cancer can be treated with compositions of the instant invention. Other hyperproliferative disorders in which affected cells express Survivin may be treated with compositions of the instant invention as well. The examples of hyperproliferative disorders that, if diagnosed to be survivin-expressing, may be treated with compositions of the instant invention include, but are not limited to, a list of solid tumors, hematologic malignancies and leukemias provided in the specification above.

The invention is also directed to methods for inhibiting angiogenesis. The instant invention teaches that down regulating expression of Survivin-ΔEx3 inhibits angiogenesis. Consequently, angiogenesis may be inhibited in a tissue by administering to the tissue compositions of the invention that down regulate the expression of Survivin-ΔEx3. Preferably, si-RNAs or sh-RNAs of the present invention comprising sequence at least 50% identical to SEQ ID NO. 24 may be used in the method. Also provided are methods of treating a condition associated with angiogenesis comprising administering to a subject in need thereof, an effective amount of a composition comprising an si-RNA or sh-RNA comprising sequence at least 50% identical to SEQ ID NO. 24. Conditions associated with angiogenesis include: the formation of solid tumors such as rhabdomyosarcomas, retinoblastomas, Ewing sarcomas, neuroblastomas and osteosarcomas; metastasis; blood born tumors such as leukemias; various acute or chronic neoplastic diseases of the bone marrow; hemangiomas; chronic inflammatory diseases; ocular neovascular disease; rheumatoid arthritis; and any other condition associated with angiogenesis known in the art.

Also provided are methods for increasing the sensitivity of a cancer cell to a chemotherapeutic agent. The instant invention teaches that the sensitivity of a cancer cell to a chemotherapeutic agent may be increased by down regulating expression of a Survivin. The method comprises administering to a cancer cell, compositions of the present invention that down regulate expression of Survivin. For example, compositions comprising an effective amount of an si-RNA or sh-RNA, wherein the si-RNA or sh-RNA comprises sequence at least 50% identical to SEQ ID NO. 24 may be administered. The chemotherapeutic agent may be taxol or any chemotherapeutic agent known in the art. The cancer cell may be resistant to the chemotherapeutic agent. Alternatively, the cancer cell may be sensitive to the chemotherapeutic agent, in which case the method may increase the sensitivity of the cancer cell to the chemotherapeutic agent. Compositions which down regulate expression of Survivin may be administered in combination with any other treatment, including treatment with the chemotherapeutic agent.

The compositions of the instant invention for treating survivin-expressing cancers and hyperproliferative disorders comprise survivin-specific si-RNAs or sh-RNAs that down regulate expression of Survivin gene by RNA interference. The si-RNAs comprise a sense strain and an antisense strain and wherein the antisense region comprises sequence complementary to an mRNA sequence encoding survivin and the sense region comprises sequence complementary to the antisense region. The compositions of the instant invention also include si-RNAs and sh-RNAs that comprise any consecutive 15 nucleotides to 35 nucleotides, and preferably 19 to 29 nucleotides complementary to Survivin having SEQ ID NO: 22, 23 or 24, as long as the selected sequence is specific to Survivin, as well as those that are at least 50% to about 99% identical to those si-RNA or sh-RNA sequences. Preferably, the si-RNA or sh-RNA compositions of the present invention comprise a 5′ and/or 3′ overhang, preferably comprising two or more nucleotides.

The compositions of the instant invention for treating survivin-expressing cancers and hyperproliferative disorders may comprise survivin-specific si-RNAs with the following sequences:

(SEQ ID NO: 1)

1). 5′- AAGCAUUCGUCCGGUUGCGUUCGUAAGCAGGCCAACGC -5′

(SEQ ID NO: 2)

2). 5′- ACUGGACAGAGAAAGAGCCUGACCUGUCUCUUUCUCGG -5′

(SEQ ID NO: 3)

3). 5′- ACUGCGAAGAAAGUGCGCCUGACGCUUCUUUCACGCGG -5′

(SEQ ID NO: 4)

4). 5′- UUUCCUUUGCAUGGGGUCGAAAGGAAACGUACCCCAGC -5′

The compositions of the instant invention may be used either as a cocktail of the four si-RNAs (SEQ ID NOs: 1, 2, 3 and 4) or as a cocktail of any combination of the si-RNAs selected from si-RNAs with SEQ ID NOs: 1, 2, 3 and 4. Alternatively, each of the si-RNA can be used separately. The si-RNAs of the instant invention may be also used in either simultaneously or sequentially combination with other chemotherapeutic agents such as for example, taxol, taxol analogs, docetaxel, docetaxel analogs and other chemotherapeutic agents known in the art as well as radiation treatment. The si-RNAs of the instant invention can also be used in a combination with other gene therapy approaches or peptide therapies. Embodiments of the instant specification may also utilize compositions comprising survivin-specific si-RNAs that are at least 50 to 99% identical to any of the si-RNAs with SEQ ID NO: 1, 2, 3 or 4 and the combinations described above.

The si-RNAs of the instant invention may be directly introduced into a cell (i.e., intracellularly); or they may be introduced extracellularly into a cavity or into the circulation system of a patient. The compositions can be introduced orally, intra-nasally or via injections. Methods for oral introduction include direct mixing of the si-RNAs with food or by preparing liquid or solid medicaments containing the si-RNAs of the instant invention. The si-RNAs of the instant invention can also be delivered via injections directly into a patient's tumor.

Instead of delivering the si-RNAs of the instant invention directly to patient's cells, expression vectors with DNA sequences encoding the si-RNAs can be used. Preferably, instead of delivering the si-RNAs of the instant invention directly to patient's cells, expression vectors with DNA sequences encoding survivin-specific sh-RNAs can be used. The DNA sequences encoding survivin-specific sh-RNAs of the instant invention comprise three regions: the first region comprises 15 to 35, preferably 19 to 29 base pair DNA sequence that at least 50%, preferably 100% identical to a region of the Survivin gene; the second region comprises a spacer DNA sequence having 5 to 20 base pairs forming the loop of the expressed RNA hair pin molecule; and the third region comprises 15 to 35, preferably 19 to 29 base pair DNA sequence at least 85% complementary and preferably 100% complementary to the first region. The second region links the first and third regions.

The first region of DNA sequences encoding survivin-specific sh-RNAs of the instant invention may comprise 15 to 35, preferably 19 to 29 base pair DNA sequence at least 50%, preferably 100% identical to SEQ ID NO. 22, 23 or 24; the second region comprises a spacer DNA sequence having 5 to 20 base pairs forming the loop of the expressed RNA hair pin molecule; and the third region comprises 15 to 35, preferably 19 to 29 base pair DNA sequence at least 85% and preferably 100% complementary to the first region.

The sh-RNA encoding DNA sequences of the instant invention can be delivered into a targeted cell by an expression vector, in which the DNA sequences are operably linked to a suitable transcriptional promoter. For example, any of the following promoters can be used: H1 RNA polymerase promoter, tRNA promoter, 7SL RNA promoter, 5 S rRNA promoter, U6 promoter.

Examples of vectors that can be used for the purposes of expressing the sh-RNAs of the instant invention in a targeted cell include plasmids, such as for example, pSuper from Oligoengine™ company, and viral vectors such as adenoviral, herpes-virus and retroviral vectors.

To prepare constructs expressing the sh-RNAs of the instant invention, the following pairs of DNA oligonucleotides can be annealed together, phosphorylated and cloned into any of the vectors of the instant invention:

(1) direct oligonucleotide for expressing sh-RNA corresponding to si-RNA with SEQ ID NO: 1 is

(SEQ ID NO: 5)

5′gatccccAAGCATTCGTCCGGTTGCGttcaagagaCGCAACCGGACGA
ATGCTTtttttggaaa
and the reverse oligonucleotide is

(SEQ ID NO: 6)

5′agcttttccaaaaaAAGCATTCGTCCGGTTGCGtctcttgaaCGCAAC
CGGACGAATGCTTggg;

(2) direct oligonucleotide for expressing sh-RNA corresponding to si-RNA with SEQ ID NO: 2 is

(SEQ ID NO: 7)

5′gatccccACTGGACAGAGAAAGAGCCttcaagagaGGCTCTTTCTCTG
TCCAGTtttttggaaa
and the reverse oligonucleotide is

(SEQ ID NO: 8)

5′agcttttccaaaaaACTGGACAGAGAAAGAGCCtctcttgaaGGCTCT
TTCTCTGTCCAGTggg;

(3) direct oligonucleotide for expressing sh-RNA corresponding to si-RNA with SEQ ID NO: 3 is

(SEQ ID NO: 9)

5′gatccccACTGCGAAGAAAGTGCGCCttcaagagaGGCGCACTTTCTT
CGCAGTtttttggaaa
and the reverse oligonucleotide is

(SEQ ID NO: 10)

5′agcttttccaaaaaACTGCGAAGAAAGTGCGCCtctcttgaaGGCGCA
CTTTCTTCGCAGTggg;

(4) direct oligonucleotide for expressing sh-RNA corresponding to si-RNA with SEQ ID NO: 4 is

(SEQ ID NO: 11)

5′GatccccTTTCCTTTGCATGGGGTCGttcaagagaCGACCCCATGCAA
AGGAAAtttttggaaa
and reverse oligonucleotide is

(SEQ ID NO: 12)

5′agcttttccaaaaaTTTCCTTTGCATGGGGTCGtctcttgaaCGACCC
CATGCAAAGGAAAggg.

In the oligonucleotides disclosed above, the survivin-specific sequences are presented in upper case letters, while hairpin and linker sequences are presented in lower case letters.

The vectors carrying coding sequences for the sh-RNAs of the instant invention can be administered orally to a patient or they can be directly injected into tumor mass if a tumor is a solid tumor. Alternatively, the vectors can be introduced into patient's blood stream intrathecally, intraperitoneally, intraventrically, intra-nasally or by any other route well known in the art.

The vectors may be admixed with other therapeutic agents such as for example, taxol, its derivative, other chemotherapeutic agent or other cancer treatment modality. The compositions can be further mixed with solvents or agents that facilitate DNA delivery into a cell, such as, for example, lipid compositions.

According to the teachings of the instant invention, a decrease of approximately 60% in the level of all survivin transcripts is sufficient to initiate apoptosis in the survivin-expressing cancer cells and substantially decrease growth of the treated tumor. It is expected that a decrease in the level of survivin transcripts of approximately 35% will be sufficient to initiate apoptosis. For example, a decrease of 35%, 40%, 45%, 50% or 55% may be sufficient to initiate apoptosis.

The present invention has multiple aspects, illustrated by the following non-limiting examples.

### EXAMPLE 1

### Analysis of Survivin Protein Expression in Pediatric Rhabdomyosarcoma Tissue Samples

Expression of survivin-protein was evaluated in 63 primary human RMS tumors that included 31 embryonal rhabdomyosarcoma (ERMS) and 32 alveolar rhabdomyosarcoma (ARMS) tumors by immunohistochemical staining with a polyclonal antibody against survivin (FL-142, Santa Cruz).

Over 80% of the tumors analyzed for survivin protein expression (54/63 tumors) had a mean survivin staining score greater than or equal to 3, indicating that at least 10 to 50% of tumor cells expressed survivin

### EXAMPLE 2

### Analysis of Survivin RNA Expression in Rhabdomyosarcoma Cell Lines

Survivin RNA expression was examined in four representative RMS cell lines by quantitative PCR. Three cell lines were alveolar in origin (CW9019, RH28 and RH30) and one was embryonal (RD2).

Total RNA was isolated from 10⁶ cells for each cell line using the Trizol® method (Invitrogen). cDNA was obtained in a random priming reaction using Omniscript reverse transcriptase (Qiagen). Primers to detect human survivin message and splice variants were designed according to the Applied Biosystem's Primer Express™ software, to quantify relative cDNA expression levels (Survivin: Forward 5′ GTG AAT TTT TGA AAC TGG ACA GAG AAA (Seq ID NO: 13); Reverse 5′ CAC TTT CTT CGC AGT TTC CTC AA (Seq ID NO: 14); Probe 5′ FAM AGC CAA GAA CAA AAT TGC AAA GGA AAC CA (Seq ID NO: 15); Survivin-2B: Forward 5′ GCA CGG TGG CTT ACG CCT G (Seq ID NO: 16); Reverse 5′ ACC GGA CGA ATG CTT TTT ATG TTC C (Seq ID NO: 17); Probe 5′ FAM ATA CCA GCA CTT TGG GAG G (Seq ID NO: 18); Survivin-ΔEx3: Forward 5′ GCT GGG AGC CAG ATG ACG (Seq ID NO: 19); Reverse 5′ TTC GCA GTT TCC TCA AAT TCT TT (Seq ID NO: 20); Probe 5′ FAM CCC CAT GCA AAG GAA ACC AAC AAT AAG AA (Seq ID NO: 21)). TaqMan analysis was carried out according to the manufacturer's instructions by using an Applied Biosystems 7700 Sequence Detection System. Results from each sample were compared with normal muscle as a calibrator using the relative standard curve method (Applied Biosystems). Genomic levels and cDNA expression levels were measured relative to 18S rRNA. Experiments were performed in triplicate and standard deviations were based on the average of three experiments.

Survivin, Survivin-2B and Survivin-ΔEx3 messages were expressed at higher levels in all four cell-lines than in their normal muscle tissue controls. Survivin RNA expression was increased 10 to 100 fold; Survivin-2B RNA expression was increased 100 to 150,000 fold, and Survivin-ΔEx3 RNA expression was 50 to 300 fold above normal muscle.

### EXAMPLE 3

### Methods of Monitoring Survivin Inhibition

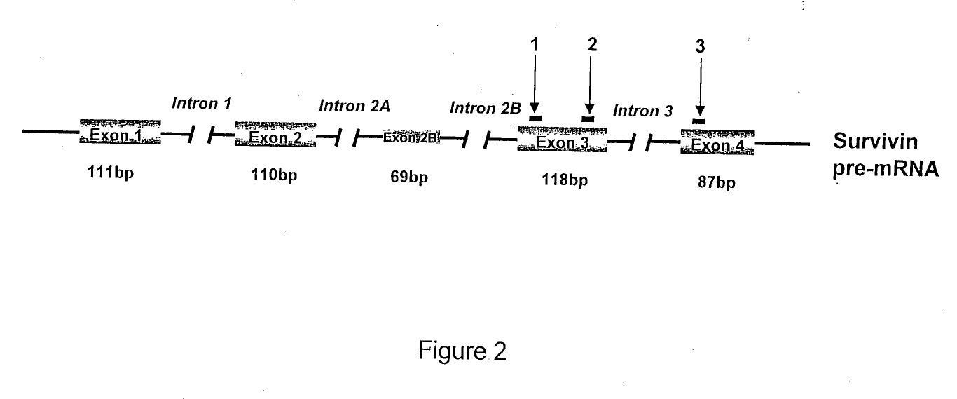

A cocktail of 3 DNA plasmids encoding survivin-specific shRNAs comprising SEQ ID NO: 1, SEQ ID NO: 2 and SEQ ID NO 3, that targeted different regions of the human survivin gene was used to eliminate survivin m-RNA in target cells (FIG. 2). The following oligonucleotides encoding the shRNAs were designed using software provided by OligoEngine company. Oligonucleotides 4 and 5 were used for the sh-RNA comprising SEQ ID NO: 1, oligonucleotides 6 and 7 were used for the sh-RNA comprising SEQ ID NO: 2 and oligonucleotides 8 and 9 were used for the sh-RNA comprising SEQ ID NO: 3. Each oligonucleotide pair was annealed, phosphorylated and cloned into Bgl II and Hind III restriction sited of pSUPER vector from OligoEngine company.

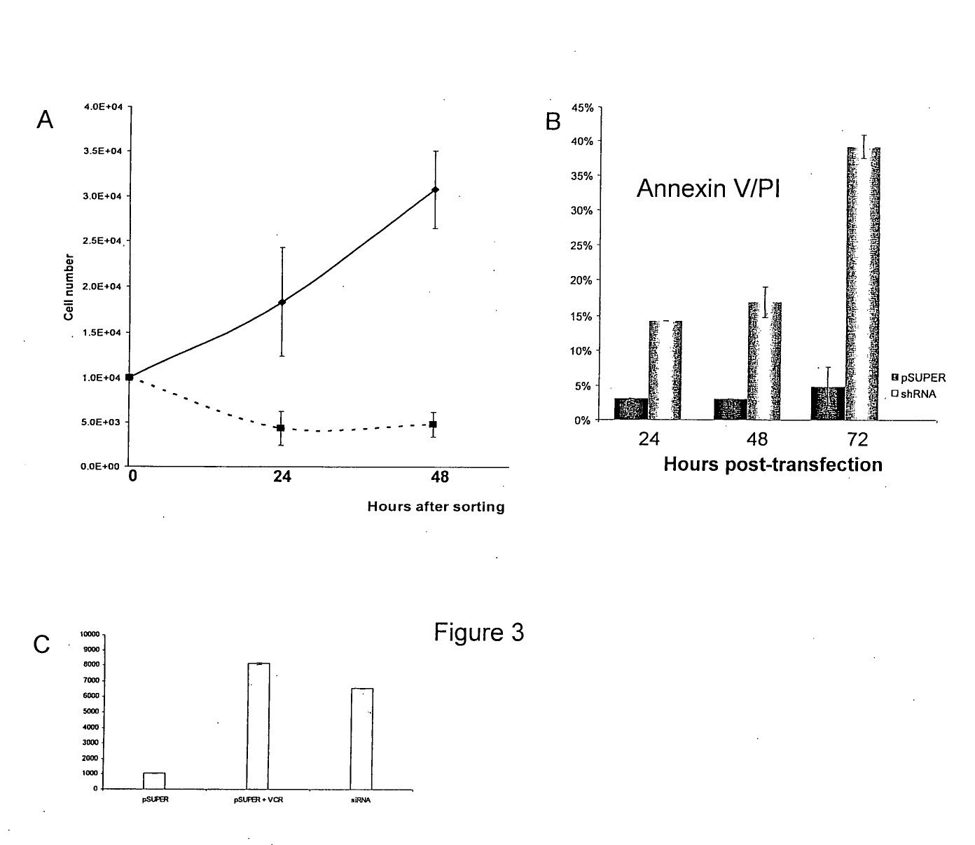

For transient transfections, plasmid DNA was transfected into proliferating CW9019 or RH30 cells using effectene transfection reagent (Qiagen) at a ratio of 1:30 (DNA:Effectene). The reporter plasmid pHcRed (Clontech) was co-transfected with pSUPER vector or a cocktail of three plasmids encoding for sh-RNA comprising SEQ ID NOs: 1, 2 and 3 into CW9019 cells. The population of cells was then sorted and enriched for transfected cells by FACS. A transfection efficiency of approximately 50% was achieved using this method, and the population of cells used for further experiments consisted of an enriched population of greater than 97% transfected cells. The growth of these cells was followed for 48 hours (FIG. 3A).

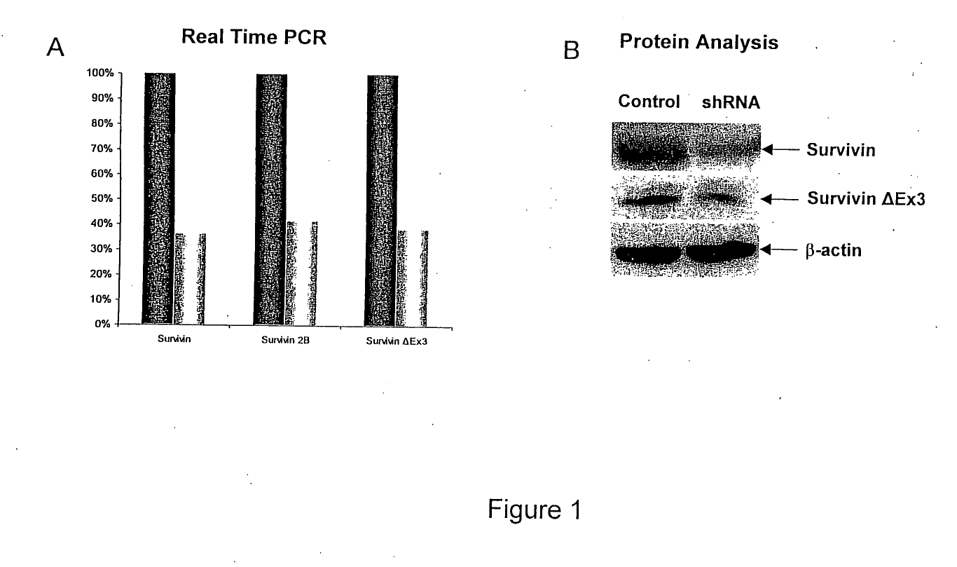

Control transfected cells grew with a doubling time of approximately 31 hours, whereas shRNA-treated cells did not double over the course of 72 h. In the shRNA-treated cells, approximately 45% of the original seeded cells were alive at 24 and 48 hours post-sorting (corresponding to 48 and 72 hours post-transfection). The same pattern of cell growth was observed in RH30 cells. These results suggest that survivin shRNAs can both inhibit cell growth and increase cell death in alveolar RMS cell lines. A decrease of approximately 60% in the levels of all Survivin transcripts, and at least a 50% reduction of survivin and survivin ΔEx3 at the protein level was observed in the RMS cells (FIGS. 1A and 1B).

To further evaluate the cell death effects implicated by the growth curves in the shRNA-treated tumor cells, an assay for early apoptosis, annexin-V/PI staining, and caspase activity assay that demonstrates involvement of the caspase pathway were performed. Annexin V/propidium iodide staining was carried out using the Roche Annexin-V-Fluos Staining Kit following the manufacturer's instructions, and analyzed by FACS in a Coulter EPICS XL flow cytometer. Measurements were performed in triplicate.

In caspase assays, two thousand cells from each experimental condition were assayed for caspase-3 and caspase-9 activity using the Caspase-Glo 3/7 Assay and Caspase-Glo 9 Assay (Promega) according to manufacturer's instructions. Caspase activity was measured in a Victor-3 plate reader (Applied Biosystems) and expressed as relative luciferase units after background subtraction. Measurements were performed in triplicate.

An increase in the percentage of apoptotic cells in shRNA-treated cells was observed at all time points analyzed (FIG. 3B). At 24 hours, 14.3% of cells were annexin positive, at 48 hours, 16.9% and at 72 hours, 39.2% were positive. This is in contrast to the 5% positivity observed in control treated cells at the 72-hour time point (FIG. 3B). Caspase 3 activity was also markedly increased in the shRNA-treated cells compared with control-treated cells at 72 hours post transfection. The level of caspase 3 activity in shRNA-treated cells was comparable to that seen following 72 hours of vincristine treatment (FIG. 3C). Caspase 9 activity was also increased in shRNA-treated cells when compared to control cells (not shown).

### EXAMPLE 4

### Tumor Engraftment Potential of Survivin si-RNA Cocktail Treated Cells

The efficiency of sh-RNA treatment for eliminating cancer cells was evaluated by assessing the potential of the sh-RNA treated RMS cells to establish subcutaneous tumors in experimental mice. To accomplish this goal, Rh30 cells were transfected ex vivo with either the sh-RNA cocktail or pSUPER control. After 24 h the cells were dislodged by trypsin digestion and 1.5×10⁶ cells injected subcutaneously to anesthetized NOD/SCID mice (n=10 per study arm). Palpable tumors were established in 9/10 control injected animals by day 15 post-inoculation. To allow for a possible slowed establishment of tumors from shRNA-treated cells the animals were followed for a period of 60 days. At the experimental end point, 9/10 control animals had very large tumors, with an average volume of 1,800 mm³. The remaining control animal, once necropsied, demonstrated an inflammatory response at the injection site. None of the animals injected with sh-RNA treated cells developed a tumor within the 60-day experimental period, or presented with signs of inflammation upon necropsy

### EXAMPLE 5

### Evaluation of the Effects of Survivin Targeted Therapy on RMS

To evaluate the effects of survivin-therapy on the RMS tumor growth in vivo, the survivin sh-RNA cocktail of the instant invention was used in treatment of a human rhabdomyosarcoma xenograft model. With this purpose, RH30^{red} cell line was established. Specifically, RH30 RMS cells were transfected with DraIII digested pDsRed2-N1 (Clontech) using Lipofectamine2000 (Invitrogen). Stably transfected cells were selected in medium containing 200 μg/ml G418 sulfate (Mediatech) for 3 weeks. Cells stably expressing DsRed protein were then expanded in basic medium and further selected based on red fluorescence during sequential sorting rounds on a Becton Dickinson FACSVantage/DiVa. RH30^{red} cells representing the brightest 20% red fluorescent cells were selected during each sterile sort. There were no apparent morphological or growth differences between the parental cell line and the red fluorescent cell population, the signature fusion gene product (PAX3-FKHR) was intact.

Alveolar rhabdomyosarcoma tumors in NOD/SCID mice were then established using an RH30^{red} cell line. For in vivo treatment studies, six-week old NOD/SCID female mice were injected with 1.5×10⁶ proliferating RH30^{red} cells subcutaneously into the right flank under isoflurane anesthesia. Approximately 21 days after inoculation 85% of the mice developed palpable red fluorescent tumors. The tumors were imaged using a fluorescent Lightools Macroimager (Lightools, Inc.), as well as measured 2 dimensionally with electronic calipers. Tumor volume was calculated using the formula

$$\frac{π}{6} \times {D}_{L} \times {D}_{s}^{2} ,$$

where D_{L}=largest diameter, and D_{S}=smallest diameter. Injections were performed hydrodynamically directly into the tumor mass, in a final volume of 100 μl, containing a total of 15 μg of DNA. Four mice per study arm were used.

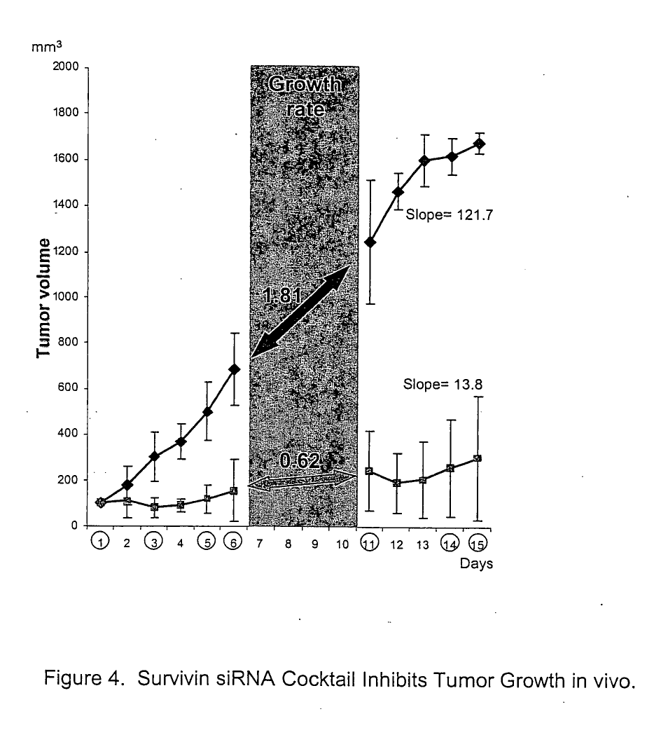

Treatment of experimental mice was initiated once the tumor volume was at least 50 mm³ (typically around 21 days post inoculation). Animals were administered the plasmid cocktail hydrodynamically, using 5 μg of each sh-RNA construct (total 15 μg) or 15μg of pSUPER control in PBS, by intratumoral injection at days 1, 3, 5, 6, 11 and 14 for a total experimental period of 15 days. Tumors from mice injected with the sh-RNA cocktail grew significantly slower than control-injected mice (FIG. 4). Differences in tumor volume were significant beginning at day 3 (48 h after the first treatment course, p<0.05). By day 4, three of four shRNA-treated animals showed a reduction in tumor size that persisted throughout the treatment period. Continuous tumor growth was observed for all control animals. Two of four control animals were sacrificed on days 11 and 12 of the experimental period due to tumor growth beyond maximum permitted growth per CRI IACUC. At the completion of the study the mean tumor volume in the control treated mice was 5.5 times greater than that of the shRNA treated mice, suggesting that the survivin sh-RNA encoding plasmid cocktail has significant antitumor activity in alveolar rhabdomyosarcoma in vivo.

At the end of the experimental period, tumors from the animals were visualized by fluorescence microscopy in vivo, then resected. The resected tumors were fixed in 10% neutral buffered formalin, and processed through an increasing ethanol series for paraffin embedding.

Tumors treated with the survivin sh-RNAs had lower levels of survivin staining than control treated tumors (16% vs 48%; p<0.0005), further supporting that the survivin sh-RNA cocktail decreased survivin protein levels in vivo. A high incidence of mitotic figures consistent with the highly proliferative nature of this type of tumor was observed in control treated mice. Tumors isolated from sh-RNA treated mice had a much lower incidence of mitotic figures (p<0.00001) and also had isolated acellular areas, suggesting loss of tumor cells in those regions due to survivin inhibition.

To investigate the etiology of the diminished tumor growth proliferation was assessed by Ki-67 immunostaining and programmed cell death was evaluated by TUNEL staining. A decrease in proliferation (9% vs 31%; p<0.005) and a small but significant increase in TUNEL staining (7.5% vs 1.9%, p=0.02) was detected in sh-RNA treated tumors, consistent with the observed growth inhibition of treated tumors

### EXAMPLE 6

### Evaluation of the Effect of Survivin ΔEx3 shRNA on Taxol-Resistant and Taxol-Sensitive Tumor Cells

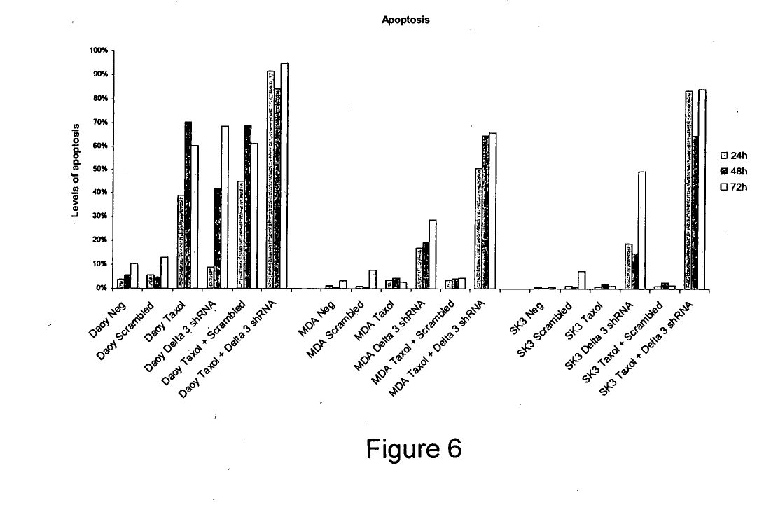

Tumor cell lines were transfected as described in Example 3 (Effectene), with scrambled shRNA or Survivin ΔEx3 shRNA comprising SEQ ID NO: 4 (PSUPER series) with or without Taxol (final concentration 20 μM). The cells were incubated for 24, 48 or 72 hours post transfection and assayed for apoptosis by Annexin V staining by flow cytometry using FloJo. The results demonstrate that tumor cells resistant to taxol treatment (MDA-MB23 1 and SKOV-3) become sensitized to taxol treatment following transfection with Survivin ΔEx3 shRNA (compare FIG. 6 “MDA Taxol” to “MDA Taxol+Delta 3 shRNA” and “SK3 Taxol” to “SK3 Taxol+Delta 3 shRNA”). Daoy cells, sensitive to taxol, demonstrate an additive effect from treatment in combination with Survivin ΔEx3 shRNA (compare FIG. 6 “Daoy Taxol” to “Daoy Taxol+Delta 3 shRNA”).

### EXAMPLE 7

### Evaluation of the Effects of Interference with Survivin ΔEx3 on Vascular Tube Formation in vitro

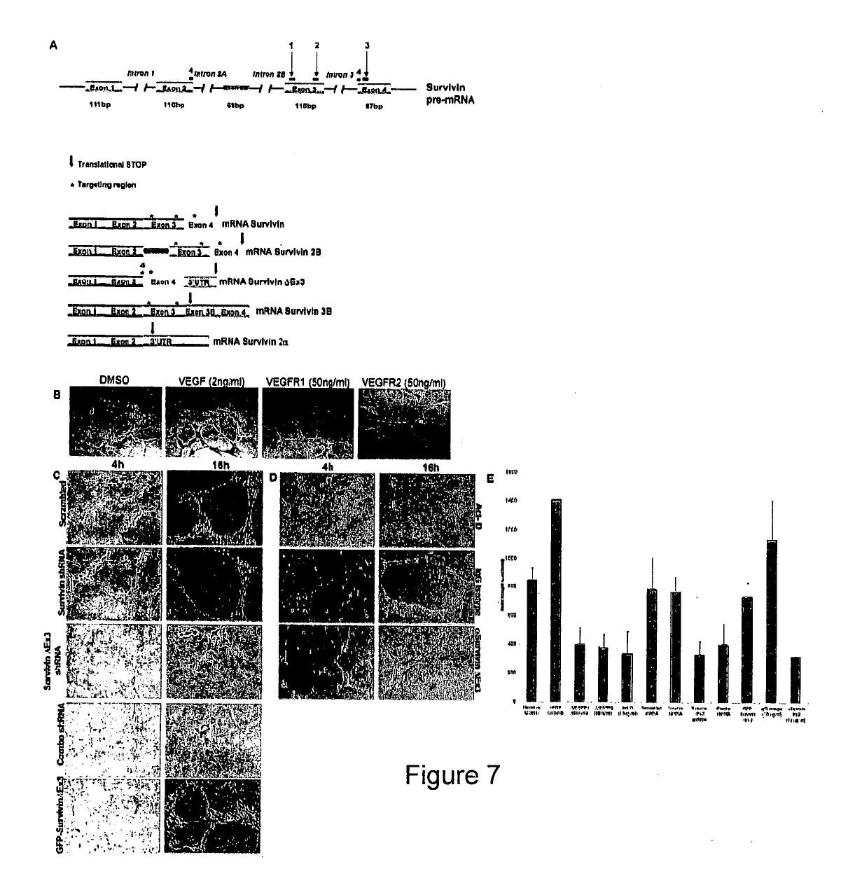

Survivin ΔEx3 activity was disrupted in an in vitro tube formation assay by either downregulating Survivin ΔEx3 mRNA with an shRNA or inhibiting Survivin ΔEx3 protein with an anti-Survivin ΔEx3 antibody. To downregulate transcription, an shRNA targeting the exon2-exon-4 boundary, specific for Survivin ΔEx3 was used. A scrambled shRNA and a cocktail of 3 shRNAs targeting all Survivin isoforms were used as controls (FIG. 7). HUVECs were transfected with shRNA plasmids in combination with a plasmid encoding eGFP. The cells were sterile sorted by FACS to isolate the population of cells that was successfully transfected, based on eGFP fluorescence. This enriched population was plated on BME with complete growth factor medium to support in vitro tube formation. FIG. 4B shows that treatment with the shRNA cocktail or Survivin ΔEx3-specific shRNA resulted in an impaired ability to form tubes on BME as compared with treatment with either a scrambled shRNA or a Survivin shRNA not targeting Survivin ΔEx3 (SH3-9). The difference was most marked between 4 and 16 hours after plating. At this point, a tubular network of interconnecting branches was well established in all control samples, however few contacts between cells, with shorter protusions and few branches were observed in cells treated with shRNA cocktail and Survivin ΔEx3-specific shRNAs. To disrupt Survivin ΔEx3 protein, HUVE cells were pre-treated with antibody specific to Survivin ΔEx3 or rabbit IgG control prior to plating on BME for in vitro tube formation. As additional controls, cells were treated with soluble VEGF, anti-VEGFR1, anti-VEGFR2 antibodies or actinomycin-D, known inducers and inhibitors of in vitro angiogenesis. Tube formation was quantified by counting the number of branches and tube length from 5 representative fields per replicate, in a double-blinded fashion. Whereas cells treated with soluble VEGF showed an increase in tube formation, cells treated with either Survivin ΔEx3 antibody, actinomycin-D, VEGFR1 or VEGFR2 antibodies showed similar abnormalities as those observed following shRNA-specific treatments, ultimately resulting in inhibition of tube formation (FIG. 7). These data suggest that Survivin ΔEx3 is required for the early steps in endothelial tube formation on BME.

### EXAMPLE 8

### Evaluation of the Effects of Interference with Survivin ΔEx3 on Directed in vivo Angiogenesis

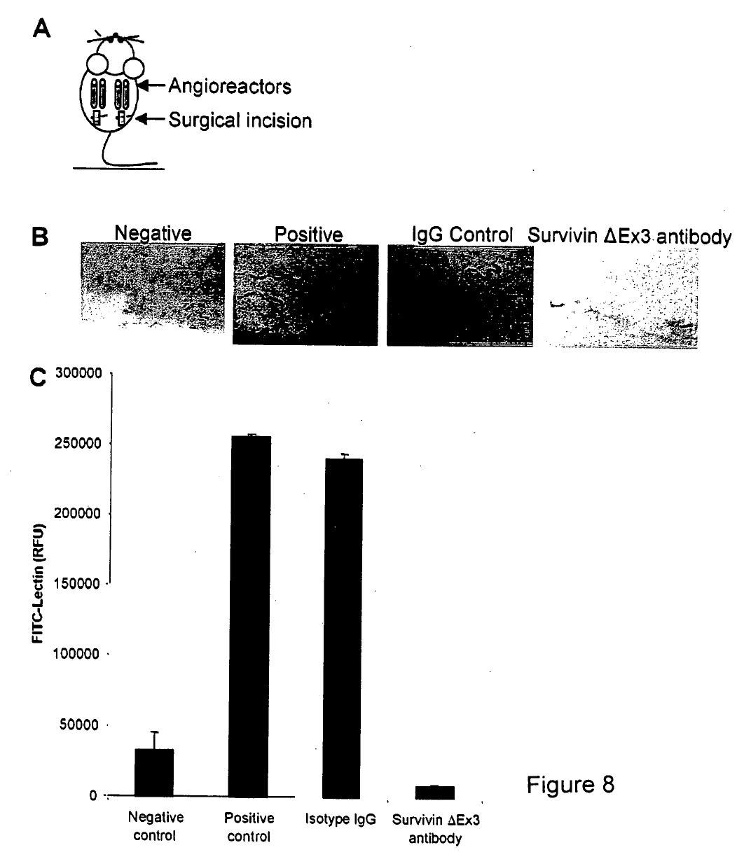

To substantiate the in vitro observation showing that Survivin ΔEx3 is required for angiogenesis, a directed in vivo angiogenesis assay (DIVAA) was performed. This assay provides a quantitative assessment of angiogenic responses to the inhibition of Survivin ΔEx3. Angioreactors filled with extracellular matrix pre-mixed with or without angiogenic factors (heparin, FGF-2) containing either normal rabbit IgG (experimental control) or Survivin ΔEx3-specific antibody were implanted subcutaneously into the dorsal flank of athymic nude mice. At a pre-determined time point known to produce significant vascular growth (11 days post-implantation), angioreactors were removed. Results at this time point showed prominent vascular growth in the control samples, as demonstrated by invasion of the angioreactor by multiple, branched capillaries (FIG. 8A). By contrast, vascularization of the angioreactor was virtually absent in the Survivin ΔEx3 antibody treated sample, similar to that observed for the negative control angioreactor (FIG. 8A). This demonstrates that inhibiting Survivin ΔEx3 can directly suppress endothelial cell invasion into the angioreactor. Fluroscein-labeled Griffonia simplicifolia lectin-1 (FITC-lectin), an endothelial cell selective marker, was used to quantify the endothelial cell responses to inhibition of Survivin ΔEx3. Endothelial cells within the angioreactors were dispersed from the matrix by dispase digestion and stained with FITC-lectin, as described. The mean relative fluorescence intensity of cells isolated form the Survivin ΔEx3 antibody-treated angioreactor was 28-fold lower than that observed for the control IgG angioreactor (FIG. 8B) this effect was more significant than the differences observed between the negative control sample lacking angiogenic modulators and the positive control sample containing angiogenic modulators (8-fold difference), representing a significantly higher level of endothelial cell invasion when Survivin ΔEx3 is not inhibited (FIG. 8B). Furthermore, a 4-fold decrease in mean intensity in the Survivin ΔEx3 antibody-treated angioreactor was observed compared to the negative control angioreactor, lacking angiogenic modulators. These data indicate that inhibition of Survivin ΔEx3 in vivo significantly reduces cell invasion, resulting in dramatically impaired angiogenesis.

### CITATIONS

Altieri D C: Validating survivin as a cancer therapeutic target. Nat Rev Cancer 3:46-54, 2003

Altieri D C: Survivin, versatile modulation of cell division and apoptosis in cancer. Oncogene 22:8581-9, 2003(a)

Altura R A, Olshefski R S, Jiang Y, et al: Nuclear expression of Survivin in paediatric ependymomas and choroid plexus tumours correlates with morphologic tumour grade. Br J Cancer 89:1743-9, 2003

Brown J M, Attardi L D. The role of apoptosis in cancer development and treatment response. Nat Rev Cancer 3:231-7, 2005.

Brummelkamp T R, Bernards R, Agami R: A system for stable expression of short interfering RNAs in mammalian cells. Science 296:550-3, 2002

Caldas H, Honsey L E, Altura R A: Survivin 2alpha: a novel Survivin splice variant expressed in human malignancies. Mol Cancer 4:11, 2005

Chen J, Jin S, Tahir S K, et al: Survivin enhances Aurora-B kinase activity and localizes Aurora-B in human cells. J Biol Chem 278:486-90, 2003

Conway E M, Pollefeyt S, Comelissen J, et al: Three differentially expressed survivin cDNA variants encode proteins with distinct antiapoptotic functions. Blood 95:1435-42, 2000

Dohi T, Beltrami E, Wall N R, et al: Mitochondrial survivin inhibits apoptosis and promotes tumorigenesis. J Clin Invest 114:1117-27, 2004

Fangusaro J, Jiang Y, Holloway M P, et al: Survivin, survivin-2B, and survivin-deltaEx3 expression in medulloblastoma: biologic markers of tumor morphology and clinical outcome. Br J Cancer 92:359-65, 2005

Fukuda S, Pelus L M: Regulation of the inhibitor-of-apoptosis family member survivin in normal cord blood and bone marrow CD34(+) cells by hematopoietic growth factors: implication of survivin expression in normal hematopoiesis. Blood 98:2091-100, 2001

Honda R, Komer R, Nigg E A: Exploring the functional interactions between Aurora B, INCENP, and survivin in mitosis. Mol Biol Cell 14:3325-41, 2003

Katayama H, Brinkley W R, Sen S: The Aurora kinases: role in cell transformation and tumorigenesis. Cancer Metastasis Rev 22:451-64, 2003

Konno R, Yamakawa H, Utsunomiya H, et al: Expression of survivin and Bcl-2 in the normal human endometrium. Mol Hum Reprod 6:529-34, 2000

Li F, Ambrosini G, Chu E Y, et al: Control of apoptosis and mitotic spindle checkpoint by survivin. Nature 396:580-4, 1998

Li F, Ackermann E J, Bennett C F, et al: Pleiotropic cell-division defects and apoptosis induced by interference with survivin function. Nat Cell Biol 1:461-6, 1999

Mahotka C, Wenzel M, Springer E, et al: Survivin-deltaEx3 and survivin-2B: two novel splice variants of the apoptosis inhibitor survivin with different antiapoptotic properties. Cancer Res 59:6097-102, 1999

Ota T, Suto S, Katayama H, et al: Increased mitotic phosphorylation of histone H3 attributable to AIM-1/Aurora-B overexpression contributes to chromosome number instability. Cancer Res 62:5168-77, 2002

Skoufias D A, Mollinari C, Lacroix F B, et al: Human survivin is a kinetochore-associated passenger protein. J Cell Biol 151:1575-82, 2000

Song Z, Yao X, Wu M: Direct interaction between survivin and Smac/DIABLO is essential for the anti-apoptotic activity of survivin during taxol-induced apoptosis. J Biol Chem 278:23130-40, 2003

Uren A G, Wong L, Pakusch M, et al: Survivin and the inner centromere protein INCENP show similar cell-cycle localization and gene knockout phenotype. Curr Biol 10:1319-28, 2000

Wheatley S P, Henzing A J, Dodson H, et al: Aurora-B phosphorylation in vitro identifies a residue of survivin that is essential for its localization and binding to inner centromere protein (INCENP) in vivo. J Biol Chem 279:5655-60, 2004

1. An si-RNA comprising a first region and a second region, wherein the first region comprises 15-35 nucleotides having a sequence at least 50% identical to a region of a survivin gene and the second region comprises 15-35 nucleotides having a sequence at least 85% complementary to the first region.

2. An sh-RNA comprising the si-RNA of claim 1, wherein the first region and the second region are linked by a spacer DNA sequence of 5-20 nucleotides to form a hairpin structure comprising a single stranded loop and a double stranded stem.

3. The sh-RNA of claim 2, wherein the survivin is encoded by a sequence selected from the group consisting of SEQ ID NOs.: 22, 23, 24 and a sequence at least 50% identical to SEQ ID NO.: 22, 23 or 24.

4. The sh-RNA of claim 2, wherein the sh-RNA comprises the sequence of SEQ ID NO.: 1, 2, 3, 4 or a sequence at least 50% identical to SEQ ID NO.: 1, 2, 3 or 4.

5. An expression vector comprising sequence encoding the sh-RNA of claim 2, operatively linked to a promoter.

6. The vector of claim 5, wherein the vector comprises sequence selected from the group consisting of: SEQ ID NOs.: 5, 6, 7, 8, 9, 10, 11 and 12.

7. A composition comprising the sh-RNA of claim 4.

8. A composition comprising the vector of claim 5.

9. The sh-RNA of claim 2, wherein the sh-RNA further comprises a 5′ and/or 3′ overhang comprising two or more nucleotides.

10. An isolated oligonucleotide comprising a sequence selected from the group consisting of SEQ ID NOs.: 13, 14, 15, 16, 17, 18, 19, 20, 21 and a nucleotide sequence at least 60% identical to any of SEQ ID NOs. 13-21.

11. A method of determining resistance of a tumor or cancer cell to chemotherapy and radiation comprising:
(a) providing a tumor or cancer cell and a healthy control cell;
(b) measuring the expression of survivin in each cell; and
(c) comparing the level of expression of survivin in the tumor or cancer cell to that in the control cell,
wherein an elevated level of expression of survivin in the tumor or cancer cell relative to the control cell indicates that the cancer cell is resistant to chemotherapy and radiation.

12. A method of determining whether a tumor or cancer is amenable to treatment with survivin-specific sh-RNA comprising:
(a) providing a sample of a patient's tumor or cancer tissue and control healthy tissue;
(b) measuring the expression of survivin in each tissue; and
(c) comparing the level of expression of survivin in the tumor or cancer tissue to that in the control tissue,
wherein an elevated level of expression of survivin in the tumor or cancer tissue relative to the control tissue indicates that the tumor or cancer is amenable to treatment with survivin-specific sh-RNA.

13. The method of claim 11 or claim 12, wherein the level of survivin expression is measured by performing quantitative PCR with the nucleic acids of claim 10.

14. The method of claim 11 or claim 12, wherein the level of survivin expression is measured by Western blotting or immunoprecipitation with antibodies specific to survivin.

15. A method of treating a hyperproliferative disease comprising administering to a subject in need thereof, an effective amount of the composition of claim 7 or claim 8.

16. A method of treating cancer comprising administering to a subject in need thereof, an effective amount of the composition of claim 7 or claim 8.

17. The method of claim 16, wherein the subject is a human.

18. The method of claim 16, wherein the composition is administered to the subject by a route selected from the group consisting of: intraperitoneal, oral, intranasal, parenteral, intrathecal, intraventricular, and injection.

19. The method of claim 16, further comprising administering to the subject taxol or any other chemotherapeutic agent.

20. A method of inhibiting angiogenesis in a tissue comprising administering to the tissue an effective amount of the composition of claim 7 or 8, wherein the survivin is encoded by SEQ ID NO. 24 or a sequence at least 50% identical to SEQ ID NO. 24.

21. A method of treating a condition associated with angiogenesis, comprising administering to a subject in need thereof, an effective amount of the composition of claim 7 or 8, wherein the survivin is encoded by SEQ ID NO. 24 or a sequence at least 50% identical to SEQ ID NO. 24.

22. A method of increasing the sensitivity of a cancer cell to a chemotherapeutic agent, comprising administering to the cancer cell an effective amount of the composition of claim 7 or 8, wherein the survivin is encoded by SEQ ID NO. 24 or a sequence at least 50% identical to SEQ ID NO. 24.

23. The method of claim 22, wherein the chemotherapeutic agent is taxol.

24. The method of claim 22, wherein the cancer cell is resistant to the chemotherapeutic agent.

25. The method of claim 22, wherein the cancer cell is not resistant to the chemotherapeutic agent.

26. A method of inhibiting the expression of survivin in cells or tissues that express survivin comprising contacting the cells or tissues with the sh-RNA of claim 4 or the composition of claim 7 or 8.

27. The method of claim 26, wherein the cells are lung cancer cells, colon cancer cells, pancreatic cancer cells, breast cancer cells, stomach cancer cells, central nervous system cancer cells, soft tissue sarcoma cells, hematologic malignant cells, pediatric cancer cells or rhabdomyosarcoma cells.

28. The method of claim 26, wherein the cells are human cells.

29. The method of claim 26, wherein the cells are stem cells, progenitor cells, bone marrow cells, vascular endothelial cells or endometrial cells.

30. The method of claim 26, wherein the tissue is fetal tissue, spleen tissue, testicular tissue or thymic tissue.

31. A method of determining whether a tissue or cell is amenable to treatment with survivin-specific sh-RNA comprising:
(a) providing a sample of a patient's cells or tissue and a control healthy cell or tissue;
(b) measuring the expression of survivin in each cell or tissue; and
(c) comparing the level of expression of survivin in said cell or tissue to that in the control cell or tissue,
wherein an elevated level of expression of survivin in said cell or tissue relative to the control cell or tissue indicates that said cell or tissue is amenable to treatment with survivin-specific sh-RNA.

32. A method of treating a condition associated with elevated survivin expression comprising administering to a subject in need thereof, an effective amount of the composition of claim 7 or 8.

33. The method of claim 32, wherein the condition is an auto-immune disease or a cardiovascular disease.

34. The method of claim 33, wherein the auto-immune disease is rheumatoid arthritis.

35. The method of claim 33, wherein the cardiovascular disease is pulmonary arterial hypertension or atherosclerosis.

36. A method of promoting apoptosis in a survivin-expressing tissue or cell, comprising administering to the tissue or cell an effective amount of the composition of claim 7 or claim 8.


SEQUENCE LISTING (26 sequences)

SEQ ID NO: 1 - RNA, 19 nt, Artificial
  SiRNA
aagcauucguccgguugcg

SEQ ID NO: 2 - RNA, 19 nt, Artificial
  SiRNA
acuggacagagaaagagcc

SEQ ID NO: 3 - RNA, 19 nt, Artificial
  SiRNA
acugcgaagaaagugcgcc

SEQ ID NO: 4 - RNA, 19 nt, Artificial
  SiRNA
uuuccuuugcauggggucg

SEQ ID NO: 5 - DNA, 64 bp, Artificial
  direct sh-RNA oligonucleotide
gatccccaagcattcgtccggttgcgttcaagagacgcaaccggacgaatgctttttttggaaa

SEQ ID NO: 6 - DNA, 64 bp, Artificial
  reverse sh-RNA oligonucleotide
agcttttccaaaaaaagcattcgtccggttgcgtctcttgaacgcaaccggacgaatgcttggg

SEQ ID NO: 7 - DNA, 64 bp, Artificial
  direct sh-RNA olignucleotide
gatccccactggacagagaaagagccttcaagagaggctctttctctgtccagttttttggaaa

SEQ ID NO: 8 - DNA, 64 bp, Artificial
  reverse sh-RNA oligonucleotide
agcttttccaaaaaactggacagagaaagagcctctcttgaaggctctttctctgtccagtggg

SEQ ID NO: 9 - DNA, 64 bp, Artificial
  direct sh-RNA oligonucleotide
gatccccactgcgaagaaagtgcgccttcaagagaggcgcactttcttcgcagttttttggaaa

SEQ ID NO: 10 - DNA, 64 bp, Artificial
  reverse sh-RNA oligonucleotide
agcttttccaaaaaactgcgaagaaagtgcgcctctcttgaaggcgcactttcttcgcagtggg

SEQ ID NO: 11 - DNA, 64 bp, Artificial
  direct sh-RNA oligonucleotide
gatcccctttcctttgcatggggtcgttcaagagacgaccccatgcaaaggaaatttttggaaa

SEQ ID NO: 12 - DNA, 64 bp, Artificial
  reverse sh-RNA oligonucleotide
agcttttccaaaaatttcctttgcatggggtcgtctcttgaacgaccccatgcaaaggaaaggg

SEQ ID NO: 13 - DNA, 27 bp, Artificial
  forward primer
gtgaatttttgaaactggacagagaaa

SEQ ID NO: 14 - DNA, 23 bp, Artificial
  reverse primer
cactttcttcgcagtttcctcaa

SEQ ID NO: 15 - DNA, 29 bp, Artificial
  probe
agccaagaacaaaattgcaaaggaaacca

SEQ ID NO: 16 - DNA, 19 bp, Artificial
  forward primer
gcacggtggcttacgcctg

SEQ ID NO: 17 - DNA, 25 bp, Artificial
  reverse primer
accggacgaatgctttttatgttcc

SEQ ID NO: 18 - DNA, 19 bp, Artificial
  probe
ataccagcactttgggagg

SEQ ID NO: 19 - DNA, 18 bp, Artificial
  forward primer
gctgggagccagatgacg

SEQ ID NO: 20 - DNA, 23 bp, Artificial
  reverse primer
ttcgcagtttcctcaaattcttt

SEQ ID NO: 21 - DNA, 29 bp, Artificial
  probe
ccccatgcaaaggaaaccaacaataagaa

SEQ ID NO: 22 - DNA, 1620 bp, Homo sapiens
ccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcggagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttc

SEQ ID NO: 23 - DNA, 1689 bp, Homo sapiens
ccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccattgggccgggcacggtggcttacgcctgtaataccagcactttgggaggccgaggcgggcggatcacgagagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcggagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttc

SEQ ID NO: 24 - DNA, 2537 bp, Homo sapiens
cccagaaggccgcggggggtggaccgcctaagagggcgtgcgctcccgacatgccccgcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatgcaaaggaaaccaacaataagaagaaagaatttgaggaaactgcggagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttcaaacactgctgtggaccctactgggtttttaaaatattgtcagtttttcatcgtcgtccctagcctgccaacagccatctgcccagacagccgcagtgaggatgagcgtcctggcagagacgcagttgtctctgggcgcttgccagagccacgaaccccagacctgtttgtatcatccgggctccttccgggcagaaacaactgaaaatgcacttcagacccacttatttctgccacatctgagtcggcctgagatagacttttccctctaaactgggagaatatcacagtggtttttgttagcagaaaatgcactccagcctctgtactcatctaagctgcttatttttgatatttgtgtcagtctgtaaatggatacttcactttaataactgttgcttagtaattggctttgtagagaagctggaaaaaaatggttttgtcttcaactcctttgcatgccaggcggtgatgtggatctcggcttctgtgagcctgtgctgtgggcagggctgagctggagccgcccctctcagcccgcctgccacggcctttccttaaaggccatccttaaaaccagaccctcatggctaccagcacctgaaagcttcctcgacatctgttaataaagccgtaggcccttgtctaagtgcaaccgcctagactttctttcagatacatgtccacatgtccatttttcaggttctctaagttggagtggagtctgggaagggttgtgaatgaggcttctgggctatgggtgaggttccaatggcaggttagagcccctcgggccaactgccatcctggaaagtagagacagcagtgcccgctgcccagaagagaccagcaagccaaactggagcccccattgcaggctgtcgccatgtggaaagagtaactcacaattgccaataaagtctcatgtggttttatctaaaaaaaaaaaaaaaaaaaaaaaaa

SEQ ID NO: 25 - DNA, 2655 bp, Homo sapiens
cccagaaggccgcggggggtggaccgcctaagagggcgtgcgctcccgacatgccccgcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcggagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttcaaacactgctgtggaccctactgggtttttaaaatattgtcagtttttcatcgtcgtccctagcctgccaacagccatctgcccagacagccgcagtgaggatgagcgtcctggcagagacgcagttgtctctgggcgcttgccagagccacgaaccccagacctgtttgtatcatccgggctccttccgggcagaaacaactgaaaatgcacttcagacccacttatttctgccacatctgagtcggcctgagatagacttttccctctaaactgggagaatatcacagtggtttttgttagcagaaaatgcactccagcctctgtactcatctaagctgcttatttttgatatttgtgtcagtctgtaaatggatacttcactttaataactgttgcttagtaattggctttgtagagaagctggaaaaaaatggttttgtcttcaactcctttgcatgccaggcggtgatgtggatctcggcttctgtgagcctgtgctgtgggcagggctgagctggagccgcccctctcagcccgcctgccacggcctttccttaaaggccatccttaaaaccagaccctcatggctaccagcacctgaaagcttcctcgacatctgttaataaagccgtaggcccttgtctaagtgcaaccgcctagactttctttcagatacatgtccacatgtccatttttcaggttctctaagttggagtggagtctgggaagggttgtgaatgaggcttctgggctatgggtgaggttccaatggcaggttagagcccctcgggccaactgccatcctggaaagtagagacagcagtgcccgctgcccagaagagaccagcaagccaaactggagcccccattgcaggctgtcgccatgtggaaagagtaactcacaattgccaataaagtctcatgtggttttatctaaaaaaaaaaaaaaaaaaaaaaaaa

SEQ ID NO: 26 - DNA, 2724 bp, Homo sapiens
cccagaaggccgcggggggtggaccgcctaagagggcgtgcgctcccgacatgccccgcggcgcgccattaaccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccattgggccgggcacggtggcttacgcctgtaataccagcactttgggaggccgaggcgggcggatcacgagagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcggagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcttgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgtttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttcaaacactgctgtggaccctactgggtttttaaaatattgtcagtttttcatcgtcgtccctagcctgccaacagccatctgcccagacagccgcagtgaggatgagcgtcctggcagagacgcagttgtctctgggcgcttgccagagccacgaaccccagacctgtttgtatcatccgggctccttccgggcagaaacaactgaaaatgcacttcagacccacttatttctgccacatctgagtcggcctgagatagacttttccctctaaactgggagaatatcacagtggtttttgttagcagaaaatgcactccagcctctgtactcatctaagctgcttatttttgatatttgtgtcagtctgtaaatggatacttcactttaataactgttgcttagtaattggctttgtagagaagctggaaaaaaatggttttgtcttcaactcctttgcatgccaggcggtgatgtggatctcggcttctgtgagcctgtgctgtgggcagggctgagctggagccgcccctctcagcccgcctgccacggcctttccttaaaggccatccttaaaaccagaccctcatggctaccagcacctgaaagcttcctcgacatctgttaataaagccgtaggcccttgtctaagtgcaaccgcctagactttctttcagatacatgtccacatgtccatttttcaggttctctaagttggagtggagtctgggaagggttgtgaatgaggcttctgggctatgggtgaggttccaatggcaggttagagcccctcgggccaactgccatcctggaaagtagagacagcagtgcccgctgcccagaagagaccagcaagccaaactggagcccccattgcaggctgtcgccatgtggaaagagtaactcacaattgccaataaagtctcatgtggttttatctaaaaaaaaaaaaaaaaaaaaaaaaa